(19) United States
(12) Patent Application Publication
(10) Pub. No.: US 2004/0156811 A1
(43) Pub. Date: Aug. 12, 2004

(54) Decorative skin and hair cosmetics containing microcrystalline cellulose as enhancing agent

(76) Inventor: Maurice Gerard Lynch, Waterloo (BE)

Correspondence Address: Patent Administrator, FMC Corporation, 1735 Market Street, Philadelphia, PA, 19103, US

(21) Appl. No.: 10/752,173

(22) Filed: Jan. 6, 2004

Related U.S. Application Data
(60) Provisional application No. 60/438,260, filed on Jan. 6, 2003.

(51) Int. Cl. A61K 7/06; A61K 7/11

(52) U.S. Cl. 424/70.13; 424/70.7

(57) ABSTRACT

The present invention is directed to decorative skin and hair cosmetics comprising an enhancing agent comprising microcrystalline cellulose. The enhancing agent enhances properties such as wrinkle-hiding, mattifying, spreading, skin-feel, physical stability of the formulation, physical stability of the cosmetic after application and the volume or curl of hair to which the cosmetic is applied. Preferred microcrystalline cellulose of the invention has an average particle size of from about 100 nanometers to about 100 microns. The microcrystalline cellulose can optionally be coprocessed with a soluble hydrocolloid during its preparation.

This application claims the benefit of U.S. Provisional Application No. 60/438,260, filed Jan. 6, 2003.

### FIELD OF THE INVENTION

The present invention is directed to the use of microcrystalline cellulose as an enhancing agent in decorative skin and hair cosmetics such as loose face powders, compact face powders, compact eyeshadows, mascaras, lipsticks, eyeliners, fluid foundations, stick foundations, etc.

### BACKGROUND OF THE INVENTION

Decorative cosmetics have been known for thousands of years and are widely used to enhance the appearance of the skin and hair. Enhancements provided by a decorative cosmetic include adding color, hiding wrinkles and increasing the volume and curl of eyelashes. In some instances, the addition of gloss is considered desirable while in other instances a mattifying effect is desirable. Many enhancing agents are known in the art. However, since consumer preferences for decorative cosmetics vary widely and are frequently changing, being dictated to a large extent by fashion trends, there is an ongoing need for new enhancing agents. Furthermore, many decorative cosmetic manufacturers market a wide range of decorative cosmetic preparations and, to minimize the number of raw materials that must be managed, there exists a need for enhancing agents that provide similar benefits in a wide range of preparations.

In addition, the acceptability of a cosmetic preparation to consumers is also determined by its stability during storage and by its properties during its application to the skin or hair. For example, if the cosmetic preparation is a liquid it is generally desirable that it should remain completely fluid and homogeneous during storage prior to use. Many of the liquid decorative cosmetics on the market at present, in particular liquid mascaras and liquid foundations, are susceptible to instability during storage. For example, liquid mascaras commonly develop ‘plugging’, a term used to describe the semi-solid layer that forms at the top of the container and needs to be broken up by vigorous movement of the brush, in up and down movements, in the bottle before the mascara can be used. Phase separation often occurs in liquid foundations and results in the appearance of an unattractive oil layer on the surface. Therefore, there exists a need for an enhancing agent that improves the stability of fluid decorative cosmetic formulations.

### SUMMARY OF THE INVENTION

The present invention is directed to decorative skin and hair cosmetics comprising an enhancing agent comprising microcrystalline cellulose. The enhancing agent enhances properties such as wrinkle-hiding, mattifying, spreading, skin-feel, physical stability of the formulation, physical stability of the cosmetic after application and the volume or curl of hairs to which the cosmetic is applied. Preferred microcrystalline cellulose of the invention has an average particle size of from about 100 nanometers to about 100 microns. The microcrystalline cellulose can optionally be co-processed with a soluble hydrocolloid during its preparation.

### DETAILED DESCRIPTION OF THE INVENTION

Microcrystalline cellulose of the invention is manufactured from cellulose. Wood pulp is a preferred source of cellulose. Microcrystalline cellulose of the invention can be divided into two categories that differ in terms of particle size, composition and method of manufacturer. These categories are non-colloidal microcrystalline cellulose and colloidal microcrystalline cellulose.

Non-colloidal microcrystalline cellulose to be used in the invention generally has an average particle size of from about 1 to about 100 microns, preferably, from about 5 to about 50 microns and most preferably from about 5 to about 30 microns. Non-colloidal microcrystalline cellulose is manufactured by a process that includes a hydrolysis step to hydrolyse the cellulose to form microcrystals and a drying step. During the drying step, extensive hydrogen bonding occurs between the microcrystals, and the resulting product consists of particles made up of several microcrytals tightly bound together. These particles cannot be easily redispersed to yield the individual microcrystals. An example of non-colloidal microcrystalline cellulose having an average particle size of about 1-100 microns is Avicel® PH105 sold by FMC Corporation, Philadelphia, Pa., having an average particle size of about 20 microns.

A second type of microcrystalline cellulose is colloidal microcrystalline cellulose. During the manufacturing process for colloidal microcrystalline cellulose a material is included that will substantially reduce hydrogen bonding during drying. Preferred materials are a soluble hydrocolloid. Preferred soluble hydrocolloids include cellulose esters, xanthan, carrageenan and alginate. A preferred cellulose ester is carboxymethylcellulose, generally referred to as cellulose gum in the cosmetic industry. Since extensive hydrogen bonding is avoided during the drying step of the manufacturing process, colloidal microcrystalline cellulose products can be readily redispersed in water to yield a network of microcrystals. Colloidal microcrystalline cellulose of the invention generally has an average particle size of less than 10 microns, preferably less than 1 micron, more preferably, between about 100 nanometers to about 1 micron. Examples of colloidal microcrystalline cellulose are Avicel® CL 611 and Avicel® PC 611 sold by FMC Corporation, Philadelphia, Pa.

Decorative cosmetic compositions of the invention include decorative loose powders, pressed powders, liquids and solids. Decorative liquid compositions include decorative pigment suspensions. Decorative solid compositions include sticks and pencils. Examples of decorative powder cosmetic compositions include facial or body foundation powder and pressed eye shadows. Examples of decorative liquid cosmetic compositions include liquid mascaras, liquid foundations, liquid eyeliner and liquid lipgloss. Examples of decorative solid cosmetic compositions include lipsticks and lipgloss sticks, pencil eyeliners and foundation sticks. Decorative cosmetic compositions of the invention can be applied to the skin, including the skin of the face and body, or hair, including the hair of the head, eyelashes and eyebrows. For example, the hair of the head can be treated with the decorative cosmetic composition of the invention to change the color of the majority of the hair or only a portion thereof.

The use of microcrystalline cellulose as a compression agent, filling agent and opacifying agent has been thought in powder cosmetics. We have now discovered that microcrystalline cellulose can be used as an enhancing agent in a range of decorative skin and hair cosmetics, including decorative powders, decorative sticks and decorative liquids. The enhancing agent of the present invention can be present in an amount effective to a act as at least one of a wrinkle-hiding agent, mattifying agent, gloss agent, spreading agent, skin-feel agent, stabilizing agent, long-lasting agent, volumizing agent or curling agent. Following are the definitions of effects provided by various agents referenced herein.

Compression agent—improves the ease of compression of cosmetic preparations, particularly difficult-to-compress materials such as pearl pigments.

Filling agent—occupies part of the volume of a cosmetic preparation.

Opacifying agent—reduces the transmission of light through a cosmetic preparation before or during application to the skin or hair.

Wrinkle-hiding agent—reduces the ability of other people to see wrinkles on the skin of a person using the cosmetic composition during normal human interaction.

Mattifying agent—reduces the shine from the skin or hair, provides a matt effect.

Gloss agent—increases the gloss of the skin or hair.

Spreading agent—improves the ease of spreading on the skin or hair and increases the homogeneity of distribution of the cosmetic preparation on the skin or hair.

Skin-feel agent—improves the perception of the cosmetic preparation on the skin during or after application.

Stabilizing agent—improves the physical stability of a cosmetic preparation before or after application to the skin or hair.

Long-lasting agent—increases the time during which the cosmetic preparation provides a desirable decorative cosmetic function on the skin or hair.

Volumizing agent—increases the perceived volume of hair.

Curling agent—increases the level of curling of hair.

Non-colloidal microcrystalline cellulose can be used as a skin-feel agent, mattifying agent, spreading agent, long-lasting agent, volumizing agent and curling agent. Examples of decorative cosmetic applications in which non-colloidal microcrystalline cellulose can be used as an enhancing agent include loose face powders, compact face powders, compact matt eye shadows, compact pearl eye shadows, eyeliners, mascaras, hair coloring preparations, lipsticks, lip gloss, fluid foundations and stick foundations. For example, in loose face powders non-colloidal microcrystalline cellulose acts as a wrinkle-hiding agent, mattifying agent, skin-feel agent and long-lasting agent. In compact face powders and compact eye shadows, non-colloidal microcrystalline cellulose acts as a spreading agent, wrinkle-hiding agent and long-lasting agent. In liquid mascaras, non-colloidal microcrystalline cellulose acts as a volumizing agent, curling agent and long-lasting agent. Without meaning to be bound by theory, it is postulated that the ability of non-colloidal microcrystalline cellulose to act as a mattifying agent is related to its porous nature that allows it to absorb sebum and oil from the skin. Thus, non-colloidal microcrystalline cellulose is particularly suited for use in decorative cosmetics intended for use by individuals with oily skin.

Colloidal microcrystalline cellulose can be used as a skin-feel agent, a gloss agent, a spreading agent and a stabilizing agent in a range of decorative cosmetics. For example, in moisturizing lipsticks, colloidal microcrystalline cellulose acts as a skin-feel agent, gloss agent and spreading agent. In liquid mascaras, colloidal microcrystalline cellulose acts as a stabilizing agent.

The present invention has particular application in decorative cosmetics for oily skin. Decorative cosmetics of the invention have enhanced properties including enhanced stability and application properties. For liquid formulations, such as mascaras, fluid foundation and fluid hair coloring products, enhanced stability includes the suspension of particles, the prevention of phase separation and the maintenance of a fluid homogenous consistency in liquid formulations. Enhanced application properties include improved ease of spreading and color distribution, reduced shine, improved skin-feel, increased gloss or matt effect and wrinkle hiding. It will be understood that these benefits are applicable to any preparation intended for use as a decorative cosmetic for skin or hair.

The cosmetics of the present invention can generally be made in accordance with conventional formulation techniques and processes, subject to the addition of the particular microcrystalline cellulose described herein. The amounts of the microcrystalline cellulose will vary depending on the application. For example, the amounts of the non-colloidal microcrystalline cellulose used in loose face powders are from about 1 to about 40%, in compact face powders and compact eyeshadows are from about 1 to about 25%, and in mascaras are from about 1 to about 20%. The amounts of the colloidal microcrystalline cellulose used in lipstick and mascara are generally from about 0.2 to about 5%.

The present invention is now described in more detail by reference to the following examples, but it should be understood that the invention is not construed as being limited thereto. Unless otherwise indicated herein, all parts, percents, ratios and the like are by weight.

### EXAMPLES

### Example 1

Loose face powder

| | | Comparative | Inventive | Inventive |
|---|---|---|---|---|
| | | Example | Example | Example |
| | | 1-1 | 1-2 | 1-3 |
| INCI Name | Tradename | % | % | % |
| Talc | | 92.8 | 88.8 | 88.8 |
| Microcrystalline | Avicel ® | | 4.0 | 0.0 |
| cellulose | pH 105 | | | |
| Microcrystalline | Avicel ® | | 0.0 | 4.0 |
| cellulose | CL 611 | | | |
| (and) cellulose | | | | |
| gum | | | | |
| Hydrogenated | | 3.0 | 3.0 | 3.0 |
| polydecene | | | | |
| Iron Oxides | | 2.9 | 2.9 | 2.9 |
| Phenoxyethanol | | 0.8 | 0.8 | 0.8 |
| Perfume | | 0.5 | 0.5 | 0.5 |
| Total | | 100.0 | 100.0 | 100.0 |

Properties of loose face powders

Comparative Example 1-1 is a typical face powder formulation and had acceptable properties in terms of skin-feel, spreading properties and appearance. Inventive Examples 1-2 and 1-3 had better skin-feel than Comparative Example 1-1 feeling softer and more velvety on the skin. In several respects, Inventive Example 1-2 was superior to both Comparative Example 1-1 and Inventive Example 1-3. Inventive Example 1-2 had the best adsorbent properties, giving a mat long-lasting effect, and was also most resistant to rub-off.

### Example 2

Compact eye shadow

| | | Comparative | Inventive |
|---|---|---|---|
| | | Example | Example |
| | | 2-1 | 2-2 |
| INCI Name | Tradename | % | % |
| Talc | | 45.2 | 34.2 |
| Microcrystalline cellulose | Avicel ® pH 105 | | 11.0 |
| Iron Oxides | | 50.0 | 50.0 |
| Hydrogenated polydecene | | 4.0 | 4.0 |
| Phenoxyethanol | | 0.8 | 0.8 |
| Total | | 100.0 | 100.0 |

Properties of compact eye shadows

Comparative Example 2-1 is a typical formulation for compact eye shadow and had acceptable properties in terms of skin-feel, pay-off and appearance. Inventive Example 2-2 had similar skin-feel and appearance and was superior in several respects. Inventive Example 2-2 had better spreading properties, produced a more even distribution of color on the skin, had better ability to hide wrinkles and had greater resistance to rub-off.

### Example 3

Mascara (oil-in-water emulsion)

| | | Comparative | Inventive | Inventive |
|---|---|---|---|---|
| | | Example | Example | Example |
| | | 3-1 | 3-2 | 3-3 |
| INCI Name | Tradename | % | % | % |
| Water | | 60.0 | 60.0 | 55.0 |
| Disodium EDTA | | 0.1 | 0.1 | 0.1 |
| Panthenol | | 0.5 | 0.5 | 0.5 |
| Glycerin | | 1.0 | 1.0 | 1.0 |
| Microcrystalline | Avicel ® | | 3.0 | 3.0 |
| cellulose | CL 611 | | | |
| (and) cellulose | | | | |
| gum | | | | |
| Microcrystalline | Avicel ® | | | 5.0 |
| cellulose | pH 105 | | | |
| Acacia Senegal | | 1.5 | | |
| Hydroxyethyl- | | 1.5 | | |
| cellulose | | | | |
| Triethanolamine | | 2.5 | 2.5 | 2.5 |
| Waxes | | 12.0 | 12.0 | 12.0 |
| Stearic acid | | 2.5 | 2.5 | 2.5 |
| Preservatives | | 1.4 | 1.4 | 1.4 |
| Iron Oxide, | | 12.0 | 12.0 | 12.0 |
| Dimethicone | | | | |
| Limnanthes alba, | | 2.5 | 2.5 | 2.5 |
| Butyrospermum | | | | |
| parkii | | | | |
| Antioxidant | | 0.5 | 0.5 | 0.5 |
| Hydrolysed | | 2.0 | 2.0 | 2.0 |
| wheat protein, | | | | |
| Hydroxypropyl- | | | | |
| siloxane | | | | |
| Total | | 100.0 | 100.0 | 100.0 |

Properties of mascaras

Comparative Example 3-1 is a typical mascara with acceptable properties in terms of application properties and appearance. Comparative Example 3-1 and Inventive Example 3-2 were very similar in several examined respects. Both were good mascaras, being easy to apply, giving definition to the lashes, and producing a medium curling and volumizing effect. Inventive Example 3-3 gave better definition to the lashes than Comparative Example 3-1 and Inventive Example 3-2. After application of Inventive Example 3-3, the lashes were more separated, longer and more curled than Comparative Example 3-1 and Inventive Example 3-2. On storage, plugging occurred around the top of the container of Comparative Example 3-1. For Inventive Examples 3-2 and 3-3, no plugging occurred. The Inventive Examples maintained a more fluid rheology than the Comparative Example.

### Example 4

Moisturizing lipsticks (water-in-oil emulsion)

| | | Comparative | Inventive |
|---|---|---|---|
| | | Example | Example |
| Ingredients | Tradename | % | % |
| Oil phase | | | |
| Castor oil | | 42.60 | 42.60 |
| Octyldodecyl ricinoleate | | 6.00 | 6.00 |
| Methyl hydrogenated rosinate | | 6.30 | 6.30 |
| Iron Oxide, Lauroyl lysine | | 7.30 | 7.30 |
| Antioxidants | | 0.85 | 0.85 |
| Preservatives | | 0.15 | 0.15 |
| Polyethylene | | 1.00 | 1.00 |
| Waxes | | 13.60 | 13.60 |
| Caprylic/capric triglyceride | | 6.00 | 6.00 |
| C10-30 Cholesterol/ | | 7.80 | 7.80 |
| lanosterol esters | | | |
| Limnanthes alba, | | 8.40 | 8.40 |
| Butyrospermum parkii | | | |
| | | 100.00 | 100.00 |
| Aqueous phase | | | |
| Arlacel 582 | | 60.00 | 60.00 |
| Preservatives | | 2.25 | 2.25 |
| Water | | 32.65 | 31.75 |
| Glycerin | | 5.00 | 5.00 |
| laluronic acid | | 0.10 | |
| Microcrystalline cellulose | Avicel ® | | 1.00 |
| | CL 611 | | |
| (and) Cellulose gum | | | |
| Total | | 100.00 | 100.00 |

In the final lipstick, the aqueous phase was present at 12% and the oil phase at 88%.

Properties of moisturizing lipsticks

Comparative Example 4-1 is a typical moisturizing lipstick of good quality. Inventive Example 4-2 was much easier to apply on the lips, had better spreadability and provided better coverage of the lips with pigment. It also had more gloss and provided more even color distribution. Inventive Example 4-2 was longer-lasting, maintaining its appearance for at least 4 hours.

While the invention has been described in detail and with reference to specific embodiments thereof, it will be apparent to one skilled in the art that various changes and modifications can be made therein without departing from the spirit and scope thereof.

What is claimed is:

1. Decorative skin and hair cosmetic compositions comprising at least one enhancing agent wherein said enhancing agent comprises microcrystalline cellulose.

2. The decorative skin and hair cosmetic composition of claim 1, wherein said microcrystalline cellulose is a non-colloidal microcrystalline cellulose having an average particle size of from about 1 micron to about 100 microns.

3. The decorative skin and hair cosmetic composition of claim 2, wherein said non-colloidal microcrystalline cellulose has an average particle size of about 20 microns.

4. The decorative skin and hair cosmetic composition of claim 1, wherein said microcrystalline cellulose is a colloidal microcrystalline cellulose having an average particle size of from about 100 nanometers to about 1 micron.

5. The decorative skin and hair cosmetic composition of claim 4, wherein said colloidal microcrystalline cellulose further comprises a soluble hydrocolloid.

6. The decorative skin and hair cosmetic compositions of claim 1, wherein said compositions are loose powders, compact powders, liquids or solids.

7. The decorative skin and hair cosmetic compositions of claim 1, wherein said compositions are mascaras, eyeliners, eyeshadow, lipgloss, lipstick, foundations, blushes, skin highlighters or hair color compositions.

8. The decorative skin and hair cosmetic compositions of claim 1, wherein said enhancing agent is present in an amount effective to act as at least one of a wrinkle-hiding agent, a mattifying agent, a gloss agent, a spreading agent, a skin-feel agent, a stabilizing agent, a long-lasting agent, a volumizing agent or a curling agent.

9. A process of preparing the decorative skin and hair cosmetic compositions in claim 1, comprising combining microcrystalline cellulose with cosmetically acceptable ingredients to form said compositions.
